(19) United States
(12) Patent Application Publication
(10) Pub. No.: US 2019/0126022 A1
(43) Pub. Date: May 2, 2019

(54) PREOPERATIVE AND POSTOPERATIVE COVER FOR EXTREMITIES

(71) Applicant: Medline Industries, Inc., Northfield, IL (US)

(72) Inventors: Michelle Christiansen, Johnsburg, IL (US); Jacob Floski, Buffalo Grove, IL (US); Jeremy Fogel, Evanston, IL (US)

(21) Appl. No.: 15/795,384

(22) Filed: Oct. 27, 2017

(51) Int. Cl. A61F 13/40

(52) U.S. Cl. CPC A61M 35/006; A61M 2209/06; A61M 2210/08; A61J 1/1425; A61M 2210/04; A61M 2207/00; A61M 2209/045

(57) ABSTRACT

Disclosed is a kit that includes a cover in the form of a boot or mitten contained within a sterile package, the kit further including a container of antiseptic liquid such as chlorhexidine gluconate. The cover is sized to fit over a human extremity (limb), and comprises an exterior layer and an interior layer, wherein the interior layer is a liquid absorbent layer and the exterior layer is a liquid impermeable layer. In use, the contents of the container of are transferred into an opening of the cover and are absorbed into the interior layer of the cover; and the cover is placed over an extremity (limb) of a human. The kit is useful for providing preoperative or postoperative sterility.

### TECHNICAL FIELD

The present disclosure relates generally to preparation of extremities for surgical procedures, in particular to preoperative and postoperative covers used to impart an antiseptic liquid to the extremities.

### BACKGROUND

When preparing a patient for a surgical procedure, it is standard for the site of the incision to be treated with an antiseptic liquid, such as chlorohexidine gluconate (CHG) solution, to reduce microbial pathogens at or near the surgical site. Typically, this chlorohexidine gluconate solution is applied to the surgical site, such as by wiping or brushing, by a member of the surgical team. A period of time is allowed to elapse prior to incision in order to allow microbial pathogens to abate. In some cases, caregivers direct patients to apply the chlorohexidine gluconate solution within a day or two before the surgical procedure is scheduled. Recent clinical studies have documented that multiple applications of 2% or 4% CHG using a standardized protocol results in high skin surface concentrations sufficient to inhibit/kill skin colonizing flora. At least one surgical study has demonstrated the efficacy of chlorhexidine gluconate administered post-operatively in reducing post-operative surgical site infection.

Skin antisepsis can be problematic at or near extremity (limb) areas of the body, and in particular on the foot or hand, where the complex morphologies of these extremities provide ample areas for microbial pathogens to reside. For example, pathogens can reside between the fingers and toes; such pathogens can be difficult to reach effectively. Further exacerbating this challenge, the areas between fingers and toes tend to be high in microbial pathogen level. In addition, the extremities can be problematic postoperatively when microbial pathogens return to the site, such as through the air or as a result of mechanical transfer. The areas between the fingers and toes provide excellent environments in which these microbial pathogens can propagate, thereby significantly increasing the risk of postoperative infection in the event these microbial pathogens are not abated.

Several embodiments of the invention advantageously address the needs above as well as other needs by providing an extremity cover in the form of a boot or mitten to use with chlorohexidine gluconate solution or other antiseptic liquid. The boot or mitten can be used in preparation of feet or hands for surgical procedures, or may be postoperatively. The boot or mitten is composed of at least an exterior layer and an interior layer, the exterior layer being at least substantially liquid impermeable and the interior layer being liquid-absorbent. The cover is provided with a container of antiseptic liquid, which is poured into an opening in the cover and allowed to absorb into the interior layer.

### BRIEF DESCRIPTION OF THE DRAWINGS

The above and other aspects, features and advantages of several embodiments of the present invention will be more apparent from the following more particular description thereof, presented in conjunction with the following drawings.

FIG. 1 is a bottom perspective view of a kit, the kit comprising a liquid impermeable package containing a preoperative or postoperative boot, and a container of chlorohexidine gluconate solution.

FIG. 2 is a top perspective view of the kit of FIG. 1, illustrates the product cover.

FIG. 3 is a top view of the kit of FIGS. 1 and 2 with the cover peeled back to reveal the contents.

FIG. 4 is a front elevational view of the container of chlorohexidine gluconate solution of FIG. 1.

FIG. 5 is a side perspective view of the boot of the kit shown in FIG. 1.

FIG. 6 is a perspective view of the boot of FIG. 5 and the container of chlorohexidine gluconate solution of FIG. 4. depicted with a cap of the container removed and with the chlorohexidine gluconate solution being poured into an opening of the boot.

FIG. 7 is a perspective view of the boot of FIG. 5 on a foot of a patient or user.

FIG. 8 is a side elevational view of the boot of FIG. 5.

FIG. 9 is a top perspective view of the boot of FIG. 5.

FIG. 10 is a perspective view of an exemplary mitten.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted to facilitate a less obstructed view of these various embodiments of the present invention.

### DETAILED DESCRIPTION

The disclosure provides a cover in the form of a boot or mitten, a kit that includes the cover and a container of an antiseptic liquid, and a number of methods, in various embodiments that are not mutually exclusive. In one embodiment, a method of treatment includes providing a cover for a human extremity (limb), the cover comprising an exterior layer and an interior layer, wherein the interior layer is a liquid absorbent layer and the exterior layer is a liquid impermeable layer; transferring contents of a container of antiseptic liquid into an opening of the cover; and placing the cover over an extremity (limb) of a human. The boot or mitten can be used at room temperature, or can be warmed for patient comfort prior to placing onto the patient's foot or hand.

In another embodiment, a method of making a kit includes providing a cover for a human extremity (limb) as heretofore described; providing a container of liquid antiseptic; and enveloping the cover for the human extremity (limb) and the container of solution in a package. The package may comprise a first part, such as a clear plastic basin, and a second part, such as a paper cover, such that the package is configured to be opened by separating the first part from the second part.

The cover may be a boot adapted for insertion of a foot through the opening, or may be a mitten adapted for insertion of a hand through the opening. No specific morphology is contemplated for the boot or cover, and it is contemplated that the boot or mitten may be sized and shaped in any appropriate way. In practice, a collection of boots or mittens, or kits containing boots or mittens, may be provided, the collection comprising boots or mittens of various sizes.

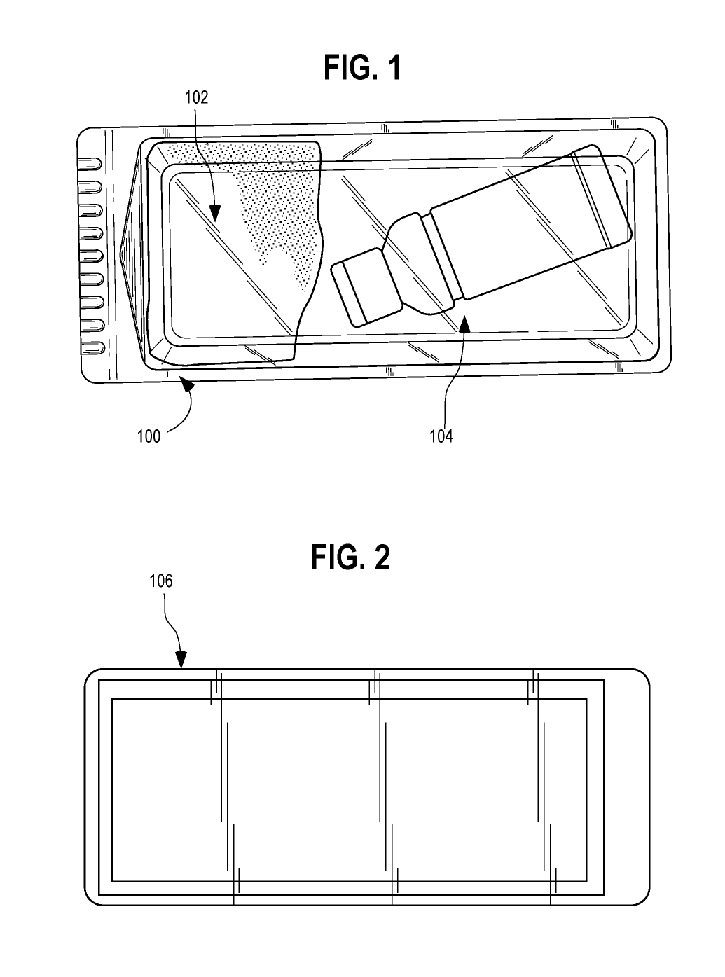

Referring first to FIG. 1, a bottom view is shown of a kit comprising a liquid impermeable package 100 containing a boot 102, and a container 104 of antiseptic liquid, which may be chlorohexidine gluconate solution. No specific morphology is contemplated for the container 104, and thus, for instance, the container may take the form of a bottle, vial, tear package, and so forth. The liquid impermeable plastic package 100 includes a peel-back cover 106. The liquid impermeable plastic package 100 preferably is a sterile package composed of a basin 105 that is at least partially transparent and the paper cover 106, the basin 105 permitting ready inspection of the contents of the package and the cover 106 being a peel- or tear-away cover that permits ready access.

The chlorohexidine gluconate solution in the container 104 may be of any suitable concentration and may be, for instance, a 2% chlorohexidine gluconate solution or 4% chlorohexidine gluconate solution. The amount of the chlorohexidine gluconate solution in the container 104 may be selected so that the entire contents of the container 104 are utilized when transferring the contents of the container 104 to the preoperative and postoperative boot 102, as described below, thereby eliminating the need to measure the amount of chlorohexidine gluconate solution per application.

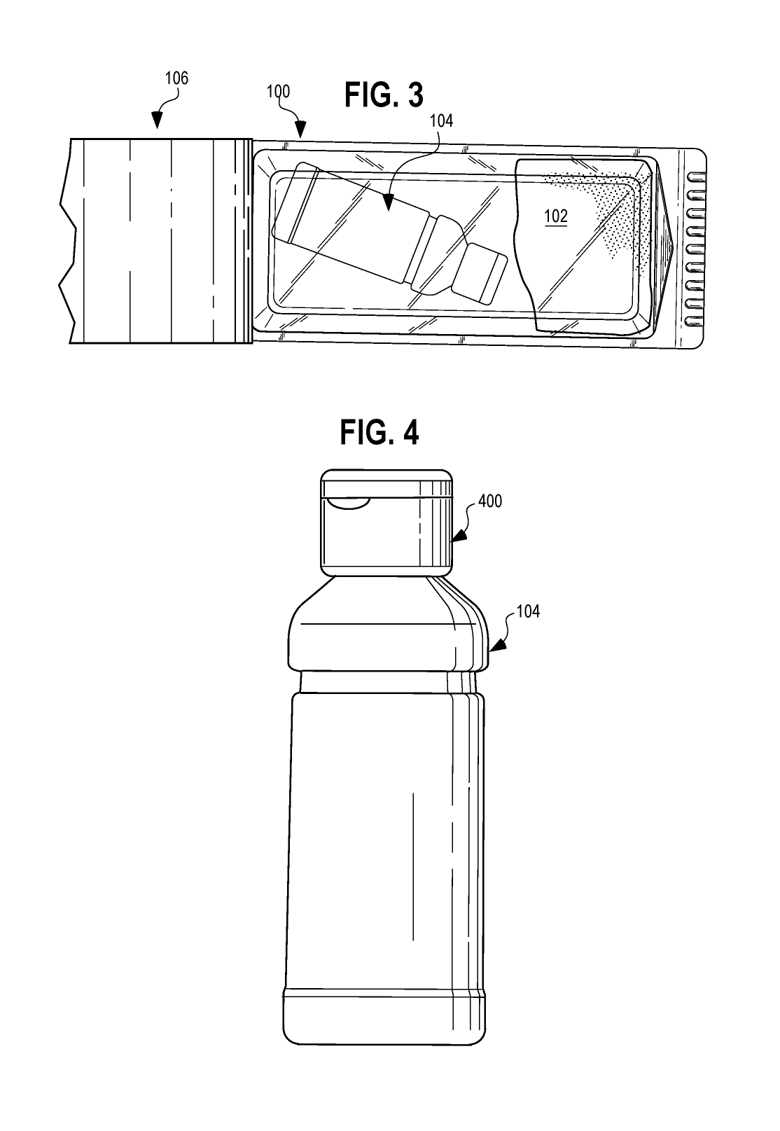

Referring next to FIGS. 2 and 3, a top view is shown of the kit of FIGS. 1 and 2 with a top paper cover peeled back 106 in FIG. 3. As seen, when the paper cover 106 is peeled back, the container 104 of chlorohexidine gluconate solution and the preoperative and postoperative boot 102 (folded) are visible. The container 104 of chlorohexidine gluconate solution is in the form a bottle having a cap 400, as shown in FIG. 4.

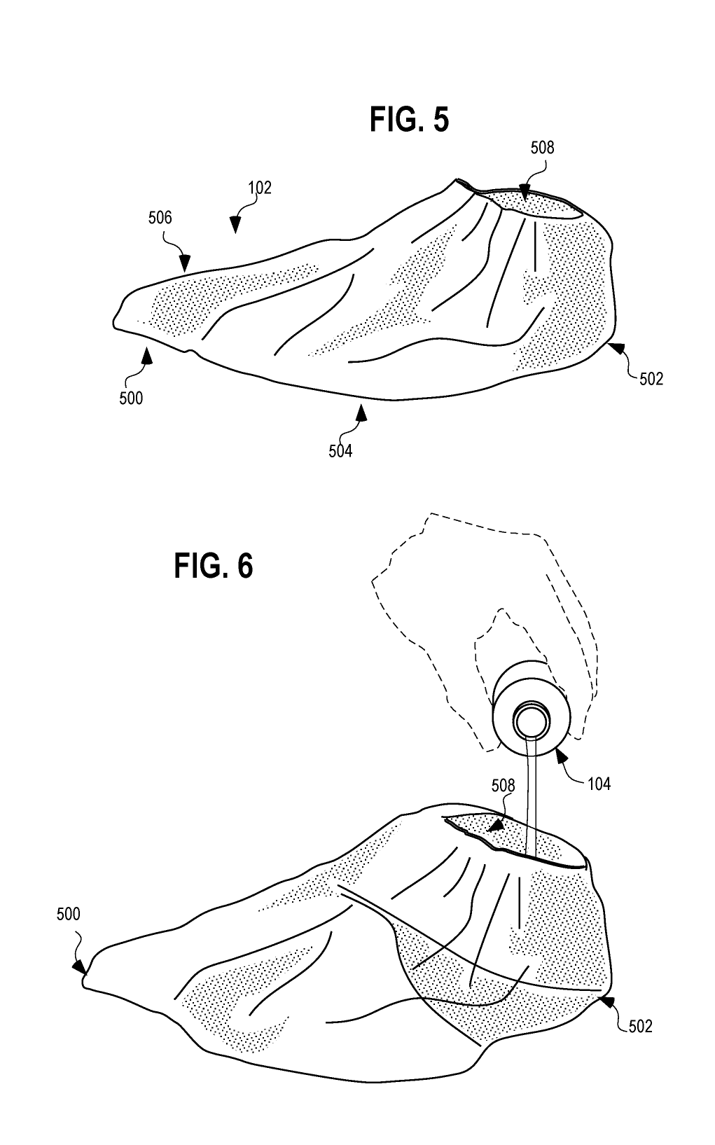

Referring to FIG. 5, the boot 102 includes a toe region 500, a heel region 502, and instep region 504, a top region 506, and an opening 508. These regions define portions of the preoperative and postoperative boot 102 that are consistent with other protective boots, booties or shoe covers designed to cover the foot 700. The boot 102 can be used on either foot 700. The toe region 500, the heel region 502, the instep region 504, and the top region 506 are all made from a layered material comprising a liquid impermeable exterior layer, and a liquid absorbent interior layer. Each layer is composed of any suitable material. For example, the interior layer may be composed of a needle punch cloth of 50% rayon and 50% polyester, while the exterior layer may be an impervious outer shell of 2.5 millimeter thick polyethylene material, such as embossed 25 gram per square meter (GSM) polyethylene sheeting with a glossy finish. These two layers can be laminated throughout their surfaces in order to provide a single structure out of which the boot is formed, or may be pinned, welded or stitched together at, for example, seams or points. The opening 508 may include elastic to secure the boot to the patient's foot 700 at the ankle, calf or thigh and to provide a barrier against escape of the contents of the boot.

Operation of the boot is shown in FIG. 6. The boot 102 is removed from the package along with a container 104 of chlorohexidine gluconate solution. Surgical personnel, medical professionals, or even the patient then transfers the contents of the container 104 of chlorohexidine gluconate solution into the opening 508 of the preoperative and postoperative boot 102, as depicted. The liquid is then quickly absorbed by the liquid absorbent inner layer inside the boot, whereupon the boot is placed over the foot of the patient.

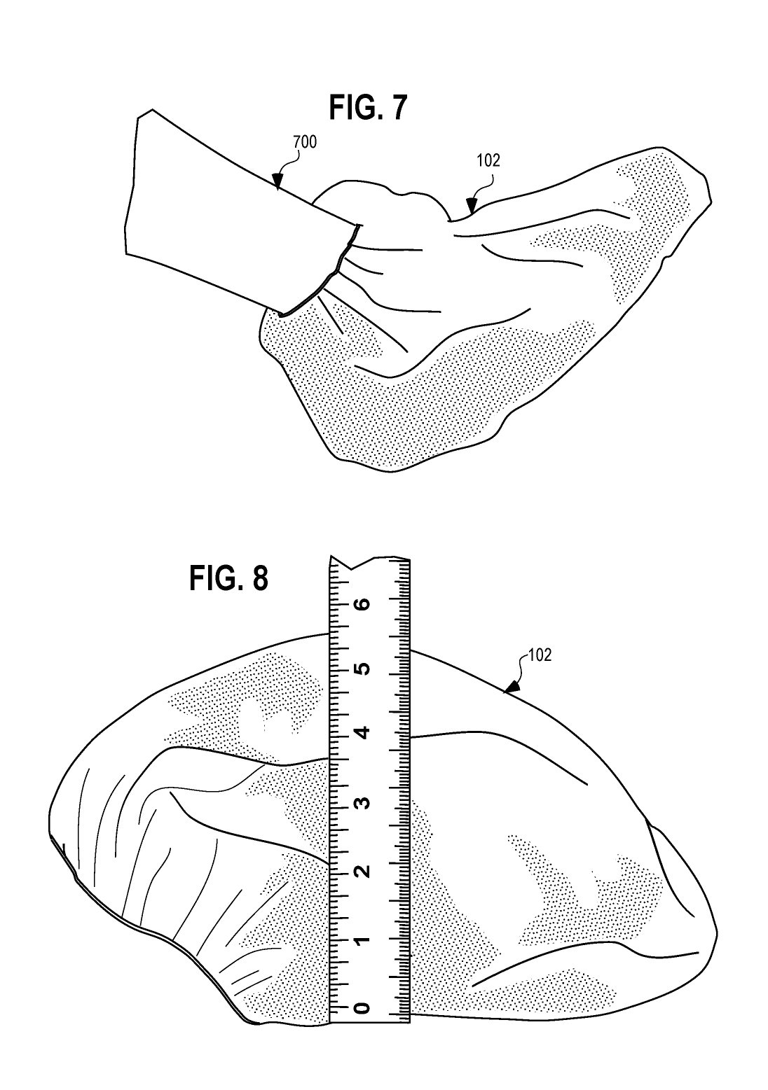

The amount of the chlorohexidine gluconate solution may be selected to fully saturate the interior layer and/or may be selected so as not to exceed an amount that the interior layer is able to absorb. Some mechanical agitation of the boot may be advantageous, such as by massaging the exterior layer of the boot with one's fingers, so as to massage the chlorohexidine gluconate solution into the interior layer in order to distribute the solution throughout and completely absorb the chlorohexidine gluconate solution into the interior layer of the boot. Advantageously, the liquid impermeable exterior layer in combination with the elasticized opening 508 prevents or inhibits the chlorohexidine gluconate solution from leaving the boot. Referring to FIG. 7, once the chlorohexidine gluconate solution is absorbed into the interior layer, the boot is pulled onto the patient's foot 700, and may be massaged into the spaces between the toes of the user in order to distribute the solution onto the entire surface of the patient's skin within the boot, or at least the surface where it is anticipated that a surgical incision will be made and an appropriate margin around the surgical incision.

In accordance with one embodiment, the patient leaves the boot in place in preparation for surgery for a period, such as 24 hours, in order that the chlorohexidine gluconate solution can continue to sterilize the surface of the patient's skin prior to surgery. Immediately prior to or a short period of time prior to surgery, such as once the patient has entered the operating room, the boot 102 may be removed from the patient. The surgery is then performed.

After surgery, the boot 102 may be returned to the patient's foot 700, or a new boot 102 may be prepared with a new container 104 of chlorohexidine gluconate solution, in the manner described above, and the boot may be placed onto the patient's foot 700 in order to reduce pathogen levels postoperatively. Preferably, the boot is a single-use boot and is discarded after preoperative use or after a period of time following surgery. The patient may be prescribed a period of time during which the preoperative and postoperative boot 102 is to remain on the patient's foot 700 following surgery, after which it can be removed and disposed of. This time may be, for example, at least 6 hours, at least 12 hours, at least 18 hours, or at least 24 hours.

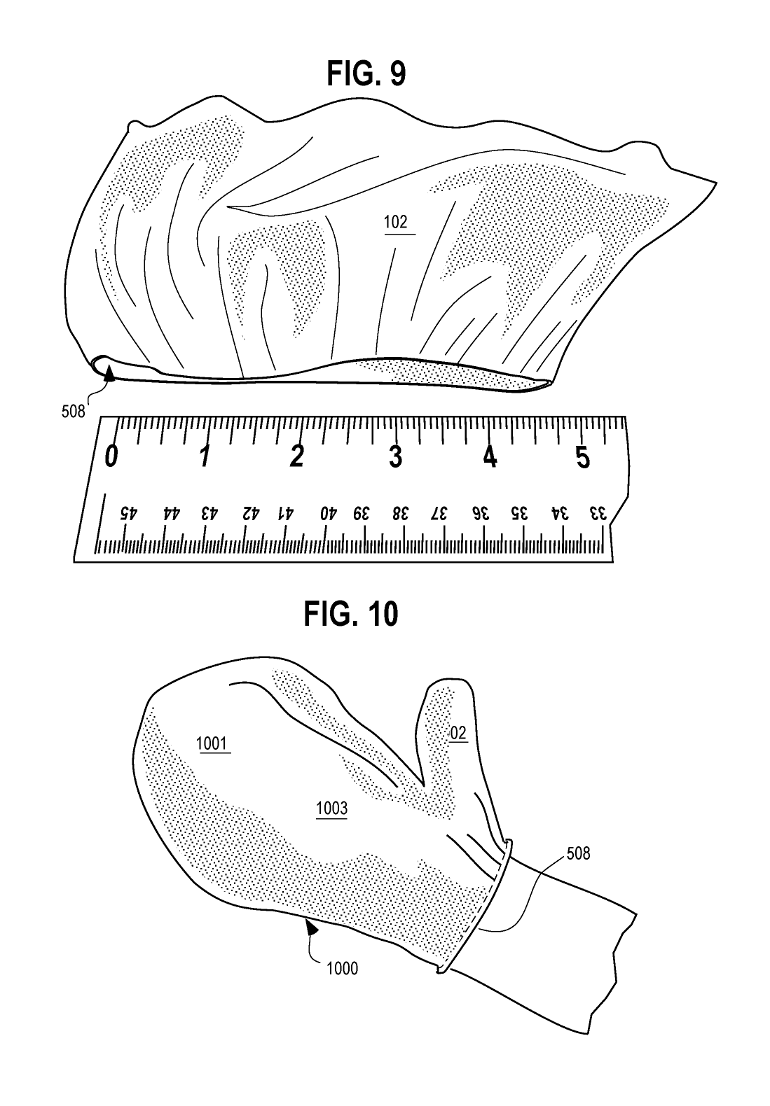

Referring to FIG. 8 a side view of the preoperative and postoperative boot 102 is shown, and referring to FIG. 9, a top partial perspective view of the preoperative and postoperative boot 102 is shown detailing an opening 508 of the preoperative and postoperative boot 102. The preoperative and postoperative boot 102 has an approximate height, from the heel region 502 to the top region 506 of 5.5 inches. The preoperative and postoperative boot 102 has an approximate length of 12.5 inches from the heel region 502 to the toe region 500. The preoperative and postoperative boot 102 opening 508 is approximately 4.5 inches to 5 inches wide (when in a relaxed state).

In an alternative embodiment, as shown in FIG. 10, the cover may be in the form of a mitten 1000 that is configured in a manner similar to that of the boot. The mitten includes a finger region 1001, thumb region 1002, palm region 1003, and wrist region 1004. As before, the mitten may be composed of a layered material comprising a liquid impermeable exterior layer, and a liquid absorbent interior layer. The mitten may be provided with an elasticized opening 508 being secured about, for example, the patient's wrist, forearm, or upper arm. The preoperative and postoperative mitten can be worn on either the right or left hand. As with the boot, the mitten 1000 is preferably a single-use, disposable product.

All methods described herein can be performed in any suitable order unless otherwise indicated herein or otherwise clearly contradicted by context. The use of any and all examples, or language describing an example (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims. Neither the marking of the patent number on any product nor the identification of the patent number in connection with any service should be deemed a representation that all embodiments described herein are incorporated into such product or service.

What is claimed is:

1. A method of treatment comprising:
providing a cover for a human hand or foot comprising an exterior layer and an interior layer, wherein the interior layer is a liquid absorbent layer and the exterior layer is a liquid impermeable layer;
transferring contents of a container of antiseptic liquid into an opening of the cover causing absorption of the liquid into the interior layer of the cover; and
placing the cover over a human hand or foot.

2. The method of claim 1 wherein said providing comprises providing a boot adapted for insertion of a foot through the opening, and wherein said placing comprises placing the boot over the foot.

3. The method of claim 2, the boot including elastic at the opening over the foot, the method comprising stretching the elastic, pulling the opening to an ankle, and relaxing the elastic around the ankle.

4. The method of claim 1 wherein said providing comprises providing a mitten adapted for insertion of a hand through the opening, and wherein said placing comprises placing the mitten over the hand.

5. The method of claim 4 wherein said mitten includes elastic at the opening over the hand, the method comprising stretching the elastic, pulling the opening to the wrist, and relaxing the elastic around the wrist.

6. The method of claim 1 wherein said absorbing is for a period of time sufficient that substantially all of the contents of the container are absorbed into the interior layer prior to said placing.

7. The method of claim 1 wherein said transferring of said contents comprises transferring an amount of said contents sufficient to saturate substantially all of the interior layer.

8. The method of claim 1, the antiseptic liquid comprising chlorohexidine gluconate.

9. The method of claim 8 wherein said placing comprises leaving the cover in place for a period of at least 24 hours.

10. The method of claim 1, wherein said interior layer comprises needle punch cloth comprising rayon and polyester.

11. The method of claim 1, wherein said exterior layer comprises polyethylene.

12. A kit comprising:
a cover for a human hand or foot comprising an exterior layer and an interior layer, wherein the interior layer is a liquid absorbent layer and the exterior layer is a liquid impermeable layer; and
a container including an antiseptic liquid;
said container and said cover being contained within a sterile package, the sterile package comprising a basin that is at least partially clear and a cover.

13. The kit of claim 12, said cover comprising a boot adapted for insertion of a foot.

14. The kit of claim 12, said cover comprising a mitten adapted for insertion of a hand.

15. The kit of claim 12, said antiseptic liquid comprising a chlorhexidine gluconate solution.

16. The kit of claim 12, wherein said interior layer comprises needle punch cloth comprising rayon and polyester.

17. The kit of claim 12, wherein said exterior layer comprises polyethylene.

18. A method of making a kit comprising:
providing a cover for a human hand or foot comprising an exterior layer and an interior layer, wherein the interior layer is a liquid absorbent layer and the exterior layer is a liquid impermeable layer;
providing a container including an antiseptic liquid;
enveloping the cover for the human hand or foot and the container of solution in a package, wherein the package comprises a first part and a second part, wherein the package is configured to be opened by separating the first part from the second part, said first part comprising a basin that is at least partially clear.

19. The method of claim 18, said container comprising a chlorohexidine gluconate solution.
